(19) United States
(12) Patent Application Publication
(10) Pub. No.: US 2023/0414240 A1
(43) Pub. Date: Dec. 28, 2023

(54) ULTRAHYDROPHOBIC COATING AND METHOD FOR LUMENS

(71) Applicants: Kester Julian Batchelor, Mound, MN (US); Teo Heng Jimmy Yang, Heath (GB)

(72) Inventors: Kester Julian Batchelor, Mound, MN (US); Teo Heng Jimmy Yang, Heath (GB)

(21) Appl. No.: 18/036,487

(22) PCT Filed: Nov. 12, 2021
(86) PCT No.: PCT/US2021/059132

§ 371 Date: May 11, 2023

Related U.S. Application Data
(60) Provisional application No. 63/113,459, filed on Nov. 13, 2020.

(51) Int. Cl. A61B 17/32; C09D 5/16; C09D 7/63; A61B 17/285

(52) U.S. Cl. CPC A61B 17/32002; C09D 5/1662; C09D 7/63; A61B 17/285; A61B 2017/320004; A61B 2017/00938

(57) ABSTRACT

A medical device and associated methods are disclosed. In one example, the medical device includes a hydrophobic coating with a hydrophobic nanoscale physical structure.

### CLAIM OF PRIORITY

This patent application claims the benefit of priority to U.S. Provisional Patent Application Ser. No. 63/113,459, entitled “ULTRAHYDROPHOBIC COATING AND METHOD FOR MEDICAL DEVICE LUMENS,” filed on Nov. 13, 2020, which is hereby incorporated by reference herein in its entirety.

### TECHNICAL FIELD

Embodiments described herein generally relate to medical devices. Specific examples of medical devices include devices that include lumens. Lumen examples include, but are not limited to, Ear, Nose, Throat (ENT) devices. Specific example devices include lumen devices that transfer tissue or fluid through a lumen, where reduced resistance and/or adhesion along lumen walls is desired.

### BACKGROUND

Several medical devices will benefit from a reduction in adhesion of material to one or more surfaces. For example in ENT devices that include a lumen, reduced adhesion can prevent clogging of gummy tissues to the internal bore. Reduced adhesion is also desired in the internal bore of any device intended to wash away fluids that may contain human tissues, such as radio frequency (RF) vaporization devices, mechanical shavers and shavers assisted with RF energy for cut and coagulation with fluid (saline) transfer etc. Improved lumen devices and other medical devices with reduced adhesion surfaces are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a debrider in accordance with some example embodiments.

FIG. 2 shows a distal end of the debrider from FIG. 1 in accordance with some example embodiments.

FIG. 3 shows a lumen portion of a medical device in accordance with some example embodiments.

FIG. 4 shows a combination of component devices including a lumen in accordance with some example embodiments.

FIG. 5 shows a hydrophobic structure of a coating on a surface of a medical device in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

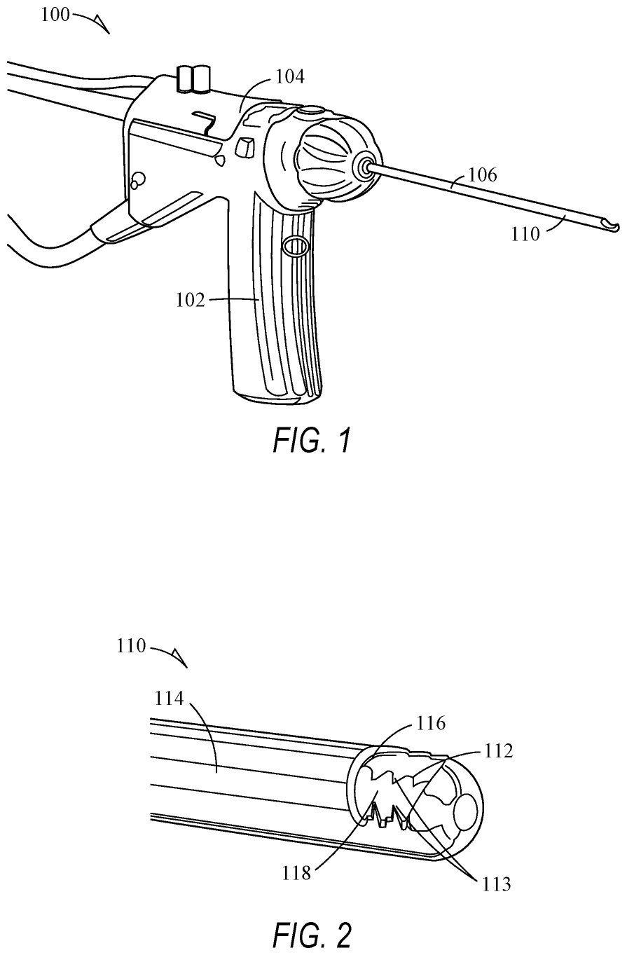

FIG. 1 illustrates an example medical device that includes a low adhesion coating according to one example. The device of FIG. 1 shows a debrider 100. The debrider 100 includes a handle 102, a body 104, and a lumen 106 having a distal end 110. The body 104 may include circuitry and actuators, such as a motor to drive blades as described in more detail below. The lumen 106 may include more than one lumen inside one another, and may include additional devices that pass through one or more lumens.

FIG. 2 shows a close up view of the distal end 110 from FIG. 1. The distal end 110 of the debrider 100 includes an outer lumen 114 and an inner lumen 112. In the example of the debrider 100, the inner lumen 112 and the outer lumen 114 cooperate to form cutting edges 113. An interface 116 is also shown between the inner lumen 112 and the outer lumen 114 where a benefit may be realized from reduced friction. An opening 118 is shown where a medium may be sucked in or expelled from one or more of the lumens 112, 114.

In examples described below, a low adhesion coating is coupled to one or more surfaces of the debrider 100. In one example, the low adhesion coating includes an ultra-hydrophobic coating. In one example, the ultra-hydrophobic coating includes a physical structure that improves hydrophobicity over other coatings that do not include such structure.

Although a debrider 100 is shown as an example in FIGS. 1 and 2, the invention is not so limited. Other example medical devices include devices with one or more lumens. Such devices include, but are not limited to, visualization devices, lasers, suction devices, infusion devices, etc.

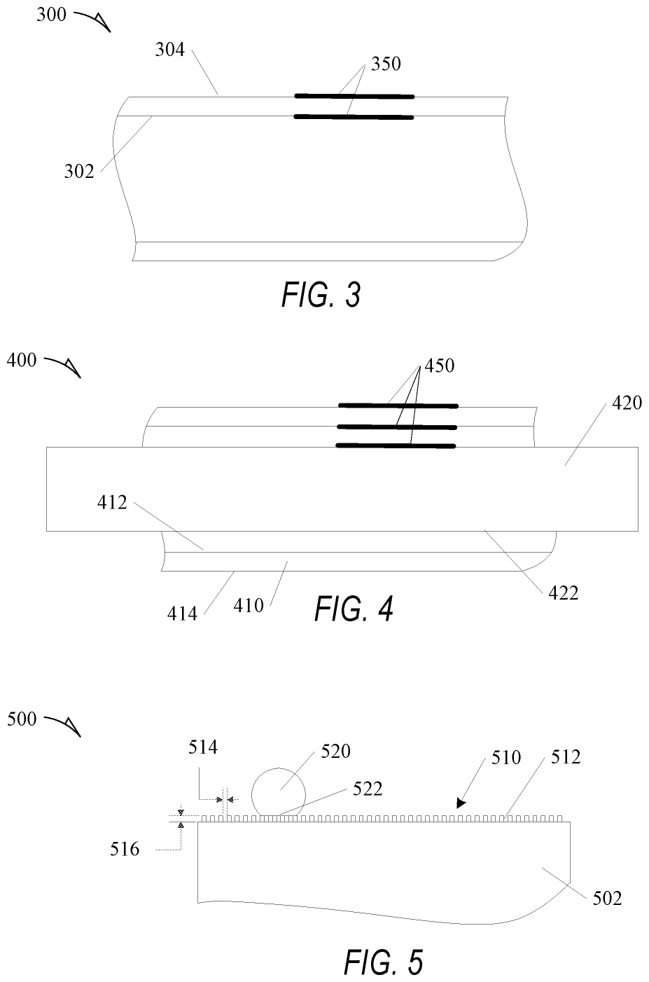

FIG. 3 shows a portion of a lumen 300, having an inner surface 302 and an outer surface 304. In one example, a low adhesion coating 350 is on a portion of one or more surfaces 302, 304. Examples of low adhesion coating 350 are described in more detail below.

FIG. 4 shows a portion of a device 400 that includes one or more lumens. An outer lumen 410 is shown with an inner surface 412 and an outer surface 414. An inner device 420, is shown within the outer lumen 410. In one example, the inner device includes a fiber optic device. In one example, the inner device includes a visualization device. In one example, the inner device includes a laser device. In one example, the inner device includes one or more tools such as forceps, blades, etc. In one example, the inner device includes an additional lumen.

In one example, a low adhesion coating 450 is on a portion of one or more surfaces 412, 414, 422. Examples of low adhesion coating 450 are described in more detail below.

FIG. 5 shows one example of a low adhesion coating 510 on a substrate 502. As shown in examples above, the coating 510 may be on all or a portion of a surface. For example, the coating 510 may be on an entire inner surface of a lumen. The coating 510 may be on only a portion of an inner surface of a lumen. The coating 510 may be on an entire outer surface of a lumen. The coating 510 may be on only a portion of an outer surface of a lumen. The coating 510 may be on both inner and outer surfaces of a lumen.

The coating 510 may be on all or a portion of a surface of an inner device.

$${Λ}_{C} = \frac{- ρ {{gV}^{1 / 3} ( ( \frac{1 - cos ( {θ}_{a} )}{sin ( {θ}_{a} )} ) ( 3 + {( \frac{1 - cos ( {θ}_{a} )}{sin ( {θ}_{a} )} )}^{2} ) )}^{2 / 3}}{{( 36 π )}^{1 / 3} γ cos ( {θ}_{a , 0} + w - 90 )}$$

where Λ is a contact line density, and Λ_{c} is a critical contact line density; p=density of the liquid droplet; g=acceleration due to gravity; V=volume of the liquid droplet; θₐ=advancing apparent contact angle; θ_{a,0}=advancing contact angle of a smooth substrate; γ=surface tension of the liquid; and w=tower wall angle.

The contact line density Λ is defined as a total perimeter of asperities over a given unit area.

In one example, if Λ>Λ_{c} then a droplet 520 of liquid are suspended in a Cassie-Baxter state. Otherwise, the droplet 520 will collapse into a Wenzel state. In one example when a Cassie-Baxter state is formed, an ultra-hydrophobic condition exists and a low adhesion coating is formed. FIG. 5 illustrates a Cassie-Baxter state, where the droplet 520 rests on top of the asperities 512. In one example, a coating thickness 516 is between 10 and 300 nanometers.

In one example, the asperities are formed by application of nanoparticles to a surface of the substrate 502. In one example, the nanoparticles include hexamethyldisiloxane (HMDSO) particles. In one example, the nanoparticles include tetramethyldisiloxane (TMDSO) particles. In one example, the nanoparticles include fluorosilane particles. Other nanoparticle materials are also within the scope of the invention. In one example, a hydrophobic chemistry of the nanoparticle, in combination with a nano scale asperity structure as shown in FIG. 5 provide better hydrophobicity compared to a hydrophobic chemistry alone.

In one example, application of appropriately sized nanoparticles provides the desired structure of asperities. In one example, etching creates all or a part of the desired structure of asperities. In one example, etching includes ion etching to form the desired structure of asperities.

Medical devices having coatings as described show reduced adhesion over other non-textured coatings for bio materials including, but not limited to, tissues, blood, fats, and/or other biological materials. This provides an anti-clogging advantage for a number of possible medical devices, including, but not limited to, ENT devices such as debriders or other infusion or aspiration devices.

To better illustrate the method and apparatuses disclosed herein, a non-limiting list of embodiments is provided here:

Example 1 includes a medical device. The medical device includes a passage for conducting a medium, and a coating on at least a portion of the passage, wherein the coating includes a hydrophobic physical structure.

Example 2 includes the medical device of example 1, wherein the coating includes hexamethyldisiloxane (HMDSO).

Example 3 includes the medical device of any one of examples 1-2, wherein the coating includes fluorosilane.

Example 4 includes the medical device of any one of examples 1-3, further including an outer lumen at least partially surrounding the passage for conducting the medium.

Example 5 includes the medical device of any one of examples 1-4, wherein the coating is located at an interface between the outer lumen and the passage for conducting the medium.

Example 6 includes the medical device of any one of examples 1 5 wherein the coating is located on an inside surface of the passage for conducting the medium.

Example 7 includes a debrider. The debrider includes an outer lumen and an inner lumen, wherein the inner lumen rotates with respect to the outer lumen, and a coating on at least a portion of one or more of the outer lumen and the inner lumen, wherein the coating includes a hydrophobic physical structure.

Example 8 includes the debrider of example 7, wherein the coating is located at an interface between the outer lumen and the inner lumen.

Example 9 includes the debrider of any one of examples 7-8, wherein the coating is located on an inside surface of the inner lumen.

Example 10 includes the debrider of any one of examples 7-9, wherein the coating is located on an outside surface of the outer lumen.

Example 11 includes the debrider of any one of examples 7-10, wherein the coating includes nanoparticles that form the hydrophobic physical structure.

Example 12 includes the debrider of any one of examples 7-11, wherein the coating includes hexamethyldisiloxane (HMDSO).

Example 13 includes the debrider of any one of examples 7-12, wherein the coating includes fluorosilane.

Example 14 includes a medical device. The medical device includes an outer lumen, an inner device, and a coating on at least a portion of one or more of the outer lumen and the inner device, wherein the coating includes a hydrophobic physical structure.

Example 15 includes the medical device of example 14, wherein the coating is located on an outside surface of the inner device.

Example 16 includes the medical device of any one of examples 14-15, wherein the coating is located on an outside surface of the outer lumen.

Example 17 includes the medical device of any one of examples 14-16, wherein the coating is located on an inside surface of the outer lumen.

Example 18 includes the medical device of any one of examples 14-17, wherein the coating includes nanoparticles that form the hydrophobic physical structure.

Example 19 includes the medical device of any one of examples 14-18, wherein the coating includes hexamethyldisiloxane (HMDSO).

Example 20 includes the medical device of any one of examples 14-19, wherein the coating includes fluorosilane.

Example 21 includes the medical device of any one of examples 14-20, wherein the inner device includes a fiber optic device.

Example 22 includes the medical device of any one of examples 14-21, wherein the inner device includes a forceps.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. A medical device, comprising:
a passage for conducting a medium; and
a coating on at least a portion of the passage, wherein the coating includes a hydrophobic physical structure.

2. The medical device of claim 1, wherein the coating includes hexamethyldisiloxane (HMDSO).

3. The medical device of claim 1, wherein the coating includes fluorosilane.

4. The medical device of claim 1, further including an outer lumen at least partially surrounding the passage for conducting the medium.

5. The medical device of claim 4, wherein the coating is located at an interface between the outer lumen and the passage for conducting the medium.

6. The medical device of claim 4, wherein the coating is located on an inside surface of the passage for conducting the medium.

7. A debrider, comprising:
an outer lumen and an inner lumen, wherein the inner lumen rotates with respect to the outer lumen; and
a coating on at least a portion of one or more of the outer lumen and the inner lumen, wherein the coating includes a hydrophobic physical structure.

8. The debrider of claim 7, wherein the coating is located at an interface between the outer lumen and the inner lumen.

9. The debrider of claim 7, wherein the coating is located on an inside surface of the inner lumen.

10. The debrider of claim 7, wherein the coating is located on an outside surface of the outer lumen.

11. The debrider of claim 7, wherein the coating includes nanoparticles that form the hydrophobic physical structure.

12. The debrider of claim 7, wherein the coating includes hexamethyldisiloxane (HMDSO).

13. The debrider of claim 7, wherein the coating includes fluorosilane.

14. A medical device, comprising:
an outer lumen;
an inner device, and
a coating on at least a portion of one or more of the outer lumen and the inner device, wherein the coating includes a hydrophobic physical structure.

15. The medical device of claim 14, wherein the coating is located on an outside surface of the inner device.

16. The medical device of claim 14, wherein the coating is located on an outside surface of the outer lumen.

17. The medical device of claim 14, wherein the coating is located on an inside surface of the outer lumen.

18. The medical device of claim 14, wherein the coating includes nanoparticles that form the hydrophobic physical structure.

19. The medical device of claim 14, wherein the coating includes hexamethyldisiloxane (HMDSO).

20. The medical device of claim 14, wherein the coating includes fluorosilane.

21. The medical device of claim 14, wherein the inner device includes a fiber optic device.

22. The medical device of claim 14, wherein the inner device includes a forceps.
